SEQUENCE LISTING (80 sequences)

SEQ ID NO: 1 - DNA, 24 bp, Artificial Sequence
  synthesis
gacagggggaggggaggagctagg

SEQ ID NO: 2 - DNA, 26 bp, Artificial Sequence
  synthesis
cttccctccaaccagttgccccaaac

SEQ ID NO: 3 - DNA, 26 bp, Artificial Sequence
  synthesis
gggaaatgggaggggtgcaaaagagg

SEQ ID NO: 4 - DNA, 26 bp, Artificial Sequence
  synthesis
ttgcgtgagtgtggatgggattggtg

SEQ ID NO: 5 - DNA, 25 bp, Artificial Sequence
  synthesis
cagccccgattcttccaccagtccc

SEQ ID NO: 6 - DNA, 25 bp, Artificial Sequence
  synthesis
cggaagattcccagtcgggttcacc

SEQ ID NO: 7 - DNA, 24 bp, Artificial Sequence
  synthesis
cagatcctaaacagctcgcagaat

SEQ ID NO: 8 - DNA, 23 bp, Artificial Sequence
  synthesis
gcgtacgcaaattaaagtccaga

SEQ ID NO: 9 - DNA, 25 bp, Artificial Sequence
  synthesis
tgctgctcacagggcccgatacttc

SEQ ID NO: 10 - DNA, 28 bp, Artificial Sequence
  synthesis
tcctttcgagctcagtgcaccacaaaac

SEQ ID NO: 11 - DNA, 24 bp, Artificial Sequence
  synthesis
gggcaagaggcaccgtcgacatca

SEQ ID NO: 12 - DNA, 27 bp, Artificial Sequence
  synthesis
gggactcggtggggctggtaacgtttc

SEQ ID NO: 13 - DNA, 28 bp, Artificial Sequence
  synthesis
gaatgctgcaaactgaccacgctggaac

SEQ ID NO: 14 - DNA, 28 bp, Artificial Sequence
  synthesis
tggcattcaagagggttttcagtctgga

SEQ ID NO: 15 - DNA, 20 bp, Artificial Sequence
  synthesis
tgcctgagaaagagaaccag

SEQ ID NO: 16 - DNA, 20 bp, Artificial Sequence
  synthesis
atggcaggatgaacaaacac

SEQ ID NO: 17 - DNA, 24 bp, Artificial Sequence
  synthesis
taccaaccaattccacaacccacc

SEQ ID NO: 18 - DNA, 23 bp, Artificial Sequence
  synthesis
atcataactccgcccattcaccg

SEQ ID NO: 19 - DNA, 22 bp, Artificial Sequence
  synthesis
aggtcggagtcaacggatttgg

SEQ ID NO: 20 - DNA, 24 bp, Artificial Sequence
  synthesis
aggctgttgtcatacttctcatgg

SEQ ID NO: 21 - DNA, 63 bp, Homo sapiens
ggctgctttagaagccattagctgcggcactgggcttggccctgccgggacttgccttgttcc

SEQ ID NO: 22 - DNA, 62 bp, Homo sapiens
ccgacgaaacttcggtaatcgacgccgtgacccgaaccgggacggccctgaacggaacaagg

SEQ ID NO: 23 - DNA, 15 bp, Homo sapiens
tgaagccattagctg

SEQ ID NO: 24 - DNA, 17 bp, Homo sapiens
gggacggccctgaacgg

SEQ ID NO: 25 - DNA, 63 bp, Homo sapiens
cgcggtgatcgagtgttggttcgtggaggatgcgagcggaaagggcctggccaagagacccgg

SEQ ID NO: 26 - DNA, 63 bp, Homo sapiens
gcgccactagctcacaaccaagcacctcctacgctcgcctttcccggaccggttctctgggcc

SEQ ID NO: 27 - DNA, 15 bp, Homo sapiens
gatcgagtgttggtt

SEQ ID NO: 28 - DNA, 18 bp, Homo sapiens
tttcccggaccggttctc

SEQ ID NO: 29 - DNA, 933 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactgggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 30 - DNA, 181 bp, Homo sapiens
gcctggcgaccgccgtctcagcaggacccgcggtgatcgagtgttggttcgtggaggatgcgagcggaaagggcctggccaagagacccggtgcactgctgttgcgccagggaccgggggaaccgccgccccggccggacctcgaccctgagctctatctcagtgtacacgaccccgcggg

SEQ ID NO: 31 - DNA, 65 bp, Homo sapiens
ggctgctttgaagccattagctgcggcactggtgggcttggccctgccgggacttgccttgttcc

SEQ ID NO: 32 - DNA, 54 bp, Homo sapiens
ggctgctttgaagccattagctgcgcttggccctgccgggacttgccttgttcc

SEQ ID NO: 33 - DNA, 65 bp, Homo sapiens
ggctgctttgaagccattagctgcggcactggggggcttggccctgccgggacttgccttgttcc

SEQ ID NO: 34 - DNA, 54 bp, Homo sapiens; identical to SEQ ID NO: 32

SEQ ID NO: 35 - DNA, 54 bp, Homo sapiens; identical to SEQ ID NO: 32

SEQ ID NO: 36 - DNA, 32 bp, Homo sapiens
gggcttggccctgccgggacttgccttgttcc

SEQ ID NO: 37 - DNA, 32 bp, Homo sapiens; identical to SEQ ID NO: 36

SEQ ID NO: 38 - DNA, 30 bp, Homo sapiens
ggctgctttgaagccattagctgcggcact

SEQ ID NO: 39 - DNA, 54 bp, Homo sapiens; identical to SEQ ID NO: 32

SEQ ID NO: 40 - DNA, 39 bp, Homo sapiens
ggctgctttgaagccattagctgcggcactgggcttgct

SEQ ID NO: 41 - DNA, 54 bp, Homo sapiens; identical to SEQ ID NO: 32

SEQ ID NO: 42 - DNA, 52 bp, Homo sapiens
ggctgctttgaagccattagctgcttggccctgccgggacttgccttgttcc

SEQ ID NO: 43 - DNA, 41 bp, Homo sapiens
ggctgctttgaagccattagctgcggcacttgccttgttcc

SEQ ID NO: 44 - DNA, 64 bp, Homo sapiens
ggctgctttgaagccattagctgcggcactgctggcttggccctgccgggacttgccttgttcc

SEQ ID NO: 45 - DNA, 65 bp, Homo sapiens
cgcggtgatcgagtgttggttcgtggagattgcgagcggaaagggcctggccaagagacccggtg

SEQ ID NO: 46 - DNA, 63 bp, Homo sapiens
cgcggtgatcgagtgttggttcgtggagagcgagcggaaagggcctggccaagagacccggtg

SEQ ID NO: 47 - DNA, 63 bp, Homo sapiens; identical to SEQ ID NO: 46

SEQ ID NO: 48 - DNA, 65 bp, Homo sapiens
cgcggtgatcgagtgttggttcgtggagatggcgagcggaaagggcctggccaagagacccggtg

SEQ ID NO: 49 - DNA, 55 bp, Homo sapiens
cgcggtgatcgagtgttggttcgtggaggaaagggcctggccaagagacccggtg

SEQ ID NO: 50 - DNA, 63 bp, Homo sapiens
cgcggtgatcgagtgttggttcgtggagtgcgagcggaaagggcctggccaagagacccggtg

SEQ ID NO: 51 - DNA, 67 bp, Homo sapiens
cgcggtgatcgagtgttggttcgtggaggatgtgcgagcggaaagggcctggccaagagacccggtg

SEQ ID NO: 52 - DNA, 64 bp, Homo sapiens
cgcggtgatcgagtgttggttcgtggaggtgcgagcggaaagggcctggccaagagacccggtg

SEQ ID NO: 53 - DNA, 62 bp, Homo sapiens
cgcggtgatcgagtgttggttcgtggatgcgagcggaaagggcctggccaagagacccggtg

SEQ ID NO: 54 - DNA, 62 bp, Homo sapiens
cgcggtgatcgagtgttggttcgtggagacgagcggaaagggcctggccaagagacccggtg

SEQ ID NO: 55 - DNA, 65 bp, Homo sapiens; identical to SEQ ID NO: 45

SEQ ID NO: 56 - DNA, 42 bp, Homo sapiens
cgcggtgatcgagcggaaagggcctggccaagagacccggtg

SEQ ID NO: 57 - DNA, 66 bp, Homo sapiens
cgcggtgatcgagtgttggttcgtggaggaatgcgagcggaaagggcctggccaagagacccggtg

SEQ ID NO: 58 - DNA, 57 bp, Homo sapiens
cgcggtgatcgagtgttggttcgtggagcggaaagggcctggccaagagacccggtg

SEQ ID NO: 59 - DNA, 66 bp, Homo sapiens; identical to SEQ ID NO: 57

SEQ ID NO: 60 - DNA, 66 bp, Homo sapiens; identical to SEQ ID NO: 57

SEQ ID NO: 61 - DNA, 62 bp, Homo sapiens
cgcggtgatcgagtgttggttcgtggaggagagcggaaagggcctggccaagagacccggtg

SEQ ID NO: 62 - DNA, 63 bp, Homo sapiens; identical to SEQ ID NO: 50

SEQ ID NO: 63 - DNA, 57 bp, Homo sapiens; identical to SEQ ID NO: 58

SEQ ID NO: 64 - DNA, 61 bp, Homo sapiens
cgcggtgatcgagtgttggttcgtggaggaagcggaaagggcctggccaagagacccggtg

SEQ ID NO: 65 - DNA, 64 bp, Homo sapiens
cgcggtgatcgagtgttggttcgtggagatgcgagcggaaagggcctggccaagagacccggtg

SEQ ID NO: 66 - DNA, 62 bp, Homo sapiens; identical to SEQ ID NO: 53

SEQ ID NO: 67 - DNA, 936 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactggtgggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 68 - DNA, 925 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcgcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 69 - DNA, 936 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactggggggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 70 - DNA, 925 bp, Homo sapiens; identical to SEQ ID NO: 68

SEQ ID NO: 71 - DNA, 925 bp, Homo sapiens; identical to SEQ ID NO: 68

SEQ ID NO: 72 - DNA, 577 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 73 - DNA, 577 bp, Homo sapiens; identical to SEQ ID NO: 72

SEQ ID NO: 74 - DNA, 356 bp, Homo sapiens
gcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcact

SEQ ID NO: 75 - DNA, 925 bp, Homo sapiens; identical to SEQ ID NO: 68

SEQ ID NO: 76 - DNA, 903 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactgggcttgctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 77 - DNA, 925 bp, Homo sapiens; identical to SEQ ID NO: 68

SEQ ID NO: 78 - DNA, 310 bp, Homo sapiens
ggctgctttgaagccattagctgcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 79 - DNA, 912 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

SEQ ID NO: 80 - DNA, 935 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactgctggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttg

(19) United States
(12) Patent Application Publication
(10) Pub. No.: US 2018/0250335 A1
(43) Pub. Date: Sep. 6, 2018

(54) MODIFIED CELLS EVOKING REDUCED IMMUNOGENIC RESPONSES

(71) Applicant: Zhejiang University, Hangzhou (CN)

(72) Inventors: Lei Xiao, Shanghai (CN); Di Cui, Hangzhou (CN)

(21) Appl. No.: 15/446,937

(22) Filed: Mar. 1, 2017

(51) Int. Cl. A61K 35/12; C07K 14/705

(52) U.S. Cl. CPC A61K 35/12; C07K 14/705

(57) ABSTRACT

Embodiments of this disclosure relate to techniques and systems for preparing modified cells evoking reduced immunogenic responses and application thereof. For example, hypo-immunogenic (e.g., decreased immunogenicity) and compatible stem cells may be obtained by a disruption in Transporter associated with antigen presentation 1 (TAP1) or TAP-associated glycoprotein (TAPBP) genes while maintaining normal pluripotency, karyotypes, and differentiation ability of these cells.

The Sequence Listing associated with this application is provided in text format in lieu of a paper copy, and is hereby incorporated by reference into the specification. The name of the text file containing the Sequence Listing is Sequence_listing.txt. The text file is about 30 KB, was created on Feb. 28, 2017, and is being submitted electronically via EFS-Web.

### BACKGROUND

Stem cells have the capacity to self-renew and differentiate. Under certain conditions, a stem cell can differentiate into a variety of functional cells. According to the stage of development, stem cells can be divided into two categories: embryonic stem cells (ES cells) and adult stem cells; according to the potential of development, stem cells can be divided into three categories: totipotent stem cells (TSC), pluripotent stem cells and unipotent stem cell. Stem cells are undifferentiated and immature with the ability to regenerate various tissues and organs of a human.

Since the 1990s, several therapy strategies involving stem cells have been attempted to cure diseases. Nevertheless, major obstacles remain to be addressed before clinical applications of stem cell-based cells replacement therapy, such as allograft immune rejection.

### SUMMARY

Described herein are techniques and systems for preparation of modified cells evoking reduced immunogenic responses. Embodiments of this disclosure relate to techniques and systems for preparing modified cells evoking reduced immunogenic responses and application thereof.

Some embodiments relate to a modified cell including a reduced amount of Major Histocompatibility Complex I (MHC I) as compared to a corresponding wild-type cell. For example, the modified cell has decreased immunogenicity as compared to the corresponding wild-type cell, and the modified cell is a modified stem cell or a cell derived from the modified stem cell.

In some embodiments, the modified stem cell has a disruption in an endogenous gene associated with a biosynthesis or transportation pathway of MHC I.

In some embodiments, the disruption may include a disruption of Transporter associated with antigen presentation 1 (TAP1) gene. For example, the disruption may include a disruption of one or more exons of TAP1 gene.

In some embodiments, the disruption of TAP1 gene may include a heterozygous disruption of TAP1 gene, and the modified cell expresses wild-type TAP1 gene.

In some embodiments, the disruption of TAP1 gene may include a homozygous disruption of TAP1 gene, and/or the modified cell does not express wild-type TAP1 gene.

In some embodiments, the disruption of the one or more exons of TAP1 gene may include a disruption of an exon of TAP1 gene having the nucleic acid sequence ID: 21 or 29. In some embodiments, the disrupted one or more exons of TAP1 gene may include one of the nucleic acid sequence IDs: 31-37, 39-44, 67-73, and 75-80. In certain embodiments, the disrupted one or more exons of TAP1 gene may include one of the nucleic acid sequence IDs: 32, 36, 37, 41, 42, 68, 72, 73, 77, and 78.

In some embodiments, the disruption may include a deletion of exon 1 of TAP1 gene, and the deletion may include the nucleic acid sequence ID: 74.

In some embodiments, the disruption may include a disruption of TAP-associated glycoprotein (TAPBP) gene. For example, a disruption of one or more exons of TAPBP gene

In some embodiments, the disruption of TAPBP gene may include a heterozygous disruption of TAP1 gene, and/or the modified cell expresses wild-type TAPBP gene.

In some embodiments, the disruption of TAPBP gene may include a homozygous disruption of TAP1 gene, and/or the modified cell does not express wild-type TAPBP gene.

In some embodiments, the disruption of the one or more exons of TAPBP gene may include a disruption of an exon of TAPBP gene having the nucleic acid sequence ID: 25 or 30. In some embodiments, the disrupted one or more exons of TAPBP gene may include one of the nucleic acid sequence IDs: 45-66. In certain embodiments, the disrupted one or more exons of TAPBP gene may include one of the nucleic acid sequence IDs: 47, 57, and 58.

In some embodiments, the decreased immunogenicity may include a decreased level of inflammatory responses induced by the modified cell as compared to the corresponding wild-type cell.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell.

In some embodiments, the MHC I is Human Leukocyte antigen I (HLA I).

In some embodiments, the modified stem cell may include at least one of a totipotent stem cell, a pluripotent stem cell, an embryonic stem cell, an induced pluripotent stem cell, or a multipotent stem cell.

In some embodiments, the modified stem cell may include a human embryonic stem cell.

Some embodiments relate to a method for generating modified stem cells for transplantation. In some embodiments, the method may include culturing in a culture media the modified cells of the embodiments described above.

Some embodiments relate to a method for reducing inflammatory responses in response to transplantation. In some embodiments, the method may include culturing in a culture media the modified cells of the embodiments described above and administering a subject a composition including the modified cells.

In some embodiments, the transplantation is an allogeneic transplantation.

In some embodiments, accumulation of T lymphocytes in the subject in response to the transplantation is reduced as compared to transplantation of wild-type cells.

In some embodiments, accumulation of NK lymphocytes in the subject response to the transplantation is reduced as compared to transplantation of wild-type cells.

Some embodiments relate to a method for improving transplantation of stem cells or cells derived from the stem cells. In some embodiments, the method may include culturing in a culture media the modified cells of the embodiments described above, and administering a subject a composition including the modified cells. For example, immune responses of the subject in response to the transplantation is less than transplantation using wild type stem cells. The subject is an animal or a human.

In some embodiments, the transplantation is an allogeneic transplantation.

In some embodiments, the immune responses comprise accumulation of T lymphocytes in the subject in response to the transplantation.

In some embodiments, the immune responses comprise accumulation of NK lymphocytes in the subject in response to the transplantation.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The detailed description is described with reference to the accompanying figures.

FIG. 1 is FIG. 1 illustrates target sites and efficiency of TALENs for transporter associated with antigen presentation 1 (TAP) and TAP-associated glycoprotein (TAPBP) in hESCs. (A) Human TAP1− TALENs targeting site at exon 1 (Up); Human TAPBP-TALENs targeting site at exon 2 (Down). (B) TALEN targeting efficiency in 293T cells in one allele. ND, not detected. NT, functional pairs but not tested. (C) Efficiency of TALENs in one or both alleles in generating TAP1− or TAPBP-deficient hESCs.

FIG. 2 shows FACS analysis of MHC class I protein expression on the cell surface in TAP1 (A) or TAPBP-(B)deficient hESC lines and wild types 24 h after IFN-y (500 U/mL) or control treatment. **p<0.05, ***p<0.01. The data are expressed as the mean±SEM.

FIG. 3a and FIG. 3b (thereafter FIG. 3) show analysis of the immunogenicity of TAP1− or TAPBP-deficient hESCs. (A) CD3-possitive T lymphocyte cells' accumulation induced by TAP1− or TAPBP-deficient hESCs and wild type hESCs. (B) NK lymphocyte marker KLRAlimmunostaining. X1 and X2 cell lines are the wild types. Quantification of the CD3+ (C) and KLRA1+ (D) cells for TAP1− and TAPBP-deficient hESCs in comparison to wild type hESCs. The number of all the nucleated cells (dyed in blue) and CD3 or KLRA1 positive cells (dyed in brown) were counted, and the ratio of them was calculated. Scale bar, 100 μm. **p<0.05, ***p<0.01. The data are expressed as the mean±SEM.

FIG. 4 shows the pluripotency of TAP1− or TAPBP-deficient hESCs. (A) RT-PCR analysis of pluripotent gene expression in TAP1− or TAPBP-deficient hESCs and the control cell (hSK: human skin cell lines). (B) RT-PCR analysis of differentiation marker expression in TAP1− or TAPBP-deficient hESCs and their embryoid bodies. U: undifferentiated; D: differentiated. (C)-D) Immunostaining of pluripotent markers in heterozygous (C) and homozygous (D) TAP1− (Left) or TAPBP-(Right) deficient hESCs. Scale bar, 100 μm. (E) Three germ layers (endoderm (Left), mesoderm (Middle), and ectoderm (Right)) of teratomas formed from the TAP1−/− or TAPBP−/− ESCs shown by HE staining. (F)-(G) Karyotype analysis of TAP1 and TAPBP heterozygous (F) and homozygous (G) hESCs.

Each of FIG. 5a and FIG. 5b (collectively “FIG. 5A”) shows a portion of TALEN targeting sequence in TAP1 (Up) and TAPBP (Down) gene locus. Each of FIG. 5c, FIG. 5d and FIG. 5e (collectively “FIG. 5B”) shows a portion of comparison of the genomic sequences of mutants and wild types at the TALEN targeting site (TAP1, Up; TAPBP, Down). Sequence identifiers for each of sequence in FIG. 5c, FIG. 5d, and FIG. 5e can be found in Table 2. FIGS. 5a, 5b, 5c, 5d, and 5e illustrate target sequence and mutants of TALENs for transporter associated with antigen presentation 1 (TAP) and TAP-associated glycoprotein (TAPBP) in hESCs. The number of deleted (dashes) or inserted (letters in red) nucleotides is labeled on the right side of each sequence in compared with the wild type sequences (wt).

FIG. 6 shows immunostaining of pluripotent markers in wild-type hESCs as a positive control. Wild-type hESCs cell lines expressed pluripotent proteins, including Nanog (A), SSEA3 (B), Oct4 (C), and Tra1-60 (D). Nuclei were stained with DAPI. Scale bar, 100 μm.

FIG. 7 shows immunostaining of pluripotent markers in CF1 cell lines as a negative control. Mouse embryonic fibroblast cell lines CF1 didn't express pluripotent proteins, including Nanog (A), SSEA3 (B), Oct4 (C), and Tra1-60 (D). Nuclei were stained with DAPI. Scale bar, 100 μm.

### DETAILED DESCRIPTION

Human embryonic stem cells (hESCs) are thought to be a promising resource for cell therapy, while it has to face the major problem of graft immunological rejection. Major histocompatibility complex (MHC) class I expressed on the cell surface is the major cause of graft rejection. However, hESCs may lose normal pluripotency, karyotypes, and differentiation ability in response to the genetic modification.

Embodiments herein relate to a surprising discovery that hypo-immunogenic (e.g., decreased immunogenicity) and compatible stem cells may be obtained by a disruption in Transporter associated with antigen presentation 1 (TAP1) or TAP-associated glycoprotein (TAPBP) genes while maintaining normal pluripotency, karyotypes, and differentiation ability of these cells. Cells differentiated from these hypoimmunogenic, and compatible stem cells may, for example, establish universal donor cells with hypo-immunogenicity.

Unless defined otherwise, all technical and scientific terms used herein have the same meaning as commonly understood by those of ordinary skill in the art to which the present disclosure belongs. Although any methods and materials similar or equivalent to those described herein can be used in the practice or testing of the present disclosure, preferred methods and materials are described.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence of a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the present disclosure. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the present disclosure. A host cell which comprises a recombinant vector of the present disclosure is a recombinant host cell.

The term “stem cell” refers to biological cells found in multicellular organisms, that can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues.

The term “immunogenicity” of a composition (e.g., a stem cell) refers to the ability of the composition to induce an immune reaction. For example, when a stem cell is transplanted to a subject, the immunogenicity may be attenuated if the stem cell does not contact MHC I and/or MHC II.

By “isolated” is meant a material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The terms “modulating” and “altering” include “increasing” and “enhancing” as well as “decreasing” or “reducing,” typically in a statistically significant or a physiologically significant amount or degree relative to a control. In specific embodiments, immunological rejection associated with transplantation of the mammalian stem cell is decreased relative to an unmodified or differently modified stem cell.

An “increased” or “enhanced” amount is typically a “statistically significant” amount, and may include an increase that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as the desired organism or a specific tissue within the desired organism. “Obtained from” can also refer to the situation in which a polynucleotide or polypeptide sequence is isolated from, or derived from, a particular organism or tissue within an organism. For example, a polynucleotide sequence encoding a reference polypeptide described herein may be isolated from a variety of prokaryotic or eukaryotic organisms, or from particular tissues or cells within a certain eukaryotic organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO2 concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt-induced), heavy metal, or an antibiotic. Numerous standard inducible promoters will be known to one of skill in the art.

The term “pluripotency” refers to the ability of ES cells that progeny cells of ES cells retain the potential for multilineage differentiation. Maintenance of pluripotency in ES cells appears to involve continual interactions between multiple nuclear factors—this achieving a balance in which some interactions are inhibitory or antagonistic, and others are positive or cooperative; as well as promoting pluripotency, this represses the genes involved in differentiation. Important factors involved in maintaining pluripotency include Oct4, Nanog, and Sox2.

The recitation “polynucleotide” or “nucleic acid” as used herein designates mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize to a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides, the term “endogenous” or “native” refers to naturally-occurring polynucleotide sequences that may be found in a given wild-type cell or organism. Also, a particular polynucleotide sequence that is isolated from a first organism and transferred to the second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The recitations “mutation” or “deletion,” in relation to the genes associated with MHC II generally refer to those changes or alterations in a stem cell that render the product of that gene non-functional or having reduced function with respect to the synthesis and/or storage of glycogen or biosynthesis of a given lipid. Examples of such changes or alterations include nucleotide substitutions, deletions, or additions to the coding or regulatory sequences of a targeted gene (e.g., CIITA), in whole or in part, which disrupt, eliminate, down-regulate, or significantly reduce the expression of the polypeptide encoded by that gene, whether at the level of transcription or translation, and/or which produce a relatively inactive (e.g., mutated or truncated) or unstable polypeptide. Techniques for producing such alterations or changes, such as by recombination with a vector having a selectable marker, are exemplified herein and known in the molecular biological art. In particular embodiments, one or more alleles of a gene, e.g., two or all alleles, may be mutated or deleted within a stem cell.

The “deletion” of a targeted gene may also be accomplished by targeting the mRNA of that gene, such as by using various antisense technologies (e.g., antisense oligonucleotides and siRNA) known in the art. Accordingly, targeted genes may be considered “non-functional” when the polypeptide or enzyme encoded by that gene is not expressed by the modified photosynthetic microorganism, or is expressed in negligible amounts, such that the modified stem cell produces or accumulates less of the polypeptide or enzyme product (e.g., MHC II) than an unmodified or differently modified stem cell.

In certain aspects, a targeted gene may be rendered “non-functional” by changes or mutations at the nucleotide level that alter the amino acid sequence of the encoded polypeptide, such that a modified polypeptide is expressed, but which has reduced function or activity (e.g., CIITA), whether by modifying that polypeptide's active site, its cellular localization, its stability, or other functional features apparent to a person skilled in the art. Such modifications to the coding sequence of a polypeptide involved in an MHC II biosynthesis or transportation pathway may be accomplished according to known techniques in the art, such as site-directed mutagenesis at the genomic level and/or natural selection (i.e., directed evolution) of a given stem cell.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In certain aspects, polypeptides may include enzymatic polypeptides, or “enzymes,” which typically catalyze various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “reference sequence” generally refers to a nucleic acid coding sequence, or amino acid sequence, to which another sequence is being compared. All polypeptide and polynucleotide sequences described herein are included as references sequences, including those described by name and those described in the Sequence Listing.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95%, 96%, 97%, 98%, 99% or greater of some given quantity.

“Transformation” refers to the permanent, heritable alteration in a cell resulting from the uptake and incorporation of foreign DNA into the host-cell genome; also, the transfer of an exogenous gene from one organism into the genome of another organism.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed in the “normal” or “wild-type” form of the gene.

Some embodiments relate to a modified cell including a reduced amount of Major Histocompatibility Complex I (MHC I) as compared to a corresponding wild-type cell. For example, the modified cell has decreased immunogenicity as compared to the corresponding wild-type cell, and the modified cell is a modified stem cell or a cell derived from the modified stem cell.

In some embodiments, the modified stem cell has a disruption in an endogenous gene associated with a biosynthesis or transportation pathway of MHC I.

Human embryonic stem cells (hESCs) are derived from inner cell mass of the human blastocyst, with the ability of self-renewal and pluripotency to differentiate into almost all cell types. As for their pluripotency, hESCs are deemed as a promising resource for the therapy of organ injury. The first clinical trial of hESCs therapy was taken in 2009: a product derived from hESCs was applied into patients for stimulating nerve growth. Afterward, stem cell clinical products became available for clinical therapy worldwide in succession.

However, immune rejection is the bottleneck which hinders the application of cell replacement therapy. Although induced pluripotent stem cells (iPSCs) can be generated from various somatic cell types, which makes it possible for personalized pluripotent stem cells, however, it is still difficult to generate iPSCs for each patient. Besides, so far, the safety concern of iPSCs is also unavoidable. Therefore, it is necessary to generate universal donor cells with low immunogenicity to make the application of cell replacement therapy more achievable.

In allograft, the major histocompatibility complex (MHC) matching between donors and recipients is a key player in the immune rejection. MHC is a set of cell surface molecules encoded by a large gene family which can determine many immune activities, such as antigen presenting, cytotoxic T cells recognition,10) and T-lymphocyte response.11) The MHC gene family is divided into three subgroups: class I, class II, and class III. In human, MHC is also called human leukocyte antigen (HLA), which mainly includes HLA I, HLA II, and HLA III subgroups.

In human allograft, when the HLA between donor and recipient is not matching, donor cells are recognized as foreign anti-gens by antigen-presenting cells (APCs) and lymphocytes of the recipient, which then trigger the immune attack, leading to the death of donor cells. Therefore, in this process, the expression of HLA on the donor cells plays a vital role in mediating allograft immune rejection. HLA I is found on the surface of almost all the nucleated cells, while HLA II is only found on APCs and lymphocytes. Thus, disrupting HLA I on the donor cells is supposed to be able to decrease the host vs. graft reaction largely and may be a way to establish universal donor cells with hypo-immunogenicity.

Currently, in order to protect the graft from immune rejection, recipients need to take a lifelong course of chemical immunosuppressive agents, which are mainly used to suppress the activation of immune system and have the risk of high toxicity, including thrombocytopenia, neurotoxicity, and carcinogenesis. Here, the modified hESCs of the present disclosure may avoid the immune rejection by escaping the recipients' immune system surveillance, instead of suppressing the whole body's defenses, which makes a better way to avoid graft immune rejection.

In the process of MHC (HLA) class I cell surface expression, firstly, cytosolic endogenous protein antigen is degraded into antigen peptides by the proteases, then the peptides are transported into endoplasmic reticulum (ER) by TAP complex, and antigen peptides are loaded on MHC class I molecules to form peptide-loading complex in ER, and finally MHC class I-peptide com-plex is presented onto the cell surface.

In some embodiments, the disruption may include a disruption of Transporter associated with antigen presentation 1 (TAP1) gene. For example, the disruption may include a disruption of one or more exons of TAP1 gene

In some embodiments, the disruption of TAP1 gene may include a heterozygous disruption of TAP1 gene, and the modified cell expresses wild-type TAP1 gene.

In some embodiments, the disruption of TAP1 gene may include a homozygous disruption of TAP1 gene, and/or the modified cell does not express wild-type TAP1 gene.

In some embodiments, the disruption of the one or more exons of TAP1 gene may include a disruption of an exon of TAP1 gene having the nucleic acid sequence ID: 21 or 29. In some embodiments, the disrupted one or more exons of TAP1 gene may include one of the nucleic acid sequence IDs: 31-37, 39-44, 67-73, and 75-80. In certain embodiments, the disrupted one or more exons of TAP1 gene may include one of the nucleic acid sequence IDs: 32, 36, 37, 41, 42, 68, 72, 73, 77, and 78.

In some embodiments, the disruption may include a deletion of exon 1 of TAP1 gene, and the deletion may include the nucleic acid sequence ID: 74.

In some embodiments, the disruption may include a disruption of TAP-associated glycoprotein (TAPBP) gene. For example, a disruption of one or more exons of TAPBP gene

In some embodiments, the disruption of TAPBP gene may include a heterozygous disruption of TAP1 gene, and the modified cell expresses wild-type TAPBP gene.

In some embodiments, the disruption of TAPBP gene may include a homozygous disruption of TAP1 gene, and/or the modified cell does not express wild-type TAPBP gene.

In some embodiments, the disruption of the one or more exons of TAPBP gene may include a disruption of an exon of TAPBP gene having the nucleic acid sequence ID: 25 or 30. In some embodiments, the disrupted one or more exons of TAPBP gene may include one of the nucleic acid sequence IDs: 45-66. In certain embodiments, the disrupted one or more exons of TAPBP gene may include one of the nucleic acid sequence IDs: 47, 57, and 58.

TAP1 and TAPBP are important genes in the regulation of MHC class I-peptide complex expression. TAP1 can mediate MHC class I-peptide complex generation by helping the delivery of cytosolic peptides into ER for their further binding to immature MHC class I molecules. Previous reports showed that TAP1 deficiency caused severe reduction of MHC class I expression and led to the failure of cytosolic antigens presenting to MHC class-I-restricted cytotoxic T lymphocytes in mice.

For example, in the human small cell lung cancer, the loss of MHC class I antigen presentation also results from the defective allele of TAP1. TAPBP, also known as tapasin, is also integral to efficient MHC class I antigen presentation and is required for the steady peptides loading of MHC class I. The previous study showed that TAPBP-mutant mice have defects in the expression of MHC class I, antigen presentation, and immune responses.

TALEN may be used to generate TAP1− and TAPBP-deficient hESC lines. These cells showed no differences with wild types in pluripotency and differentiation abilities. However, FACS data indicated that HLA I is largely reduced on the cell surface in the deficient hESCs. Compared with wild type, the injection of TAP1− or TAPBP-deficient cells into mice induced significantly reduced immunological rejection. Therefore, the TAP1− or TAPBP-deficient cells might be a promising resource for clinical use.

In some embodiments, the decreased immunogenicity may include a decreased level of inflammatory responses induced by the modified cell as compared to the corresponding wild-type cell.

In some embodiments, a karyotype of the modified cell is the same as a karyotype of the corresponding wild-type cell.

In some embodiments, a level of pluripotency of the modified cell is substantially the same as a level of pluripotency of the corresponding wild-type cell.

In some embodiments, the MHC I is Human Leukocyte antigen I (HLA I).

In some embodiments, the modified stem cell may include at least one of a totipotent stem cell, a pluripotent stem cell, an embryonic stem cell, an induced pluripotent stem cell, or a multipotent stem cell.

In some embodiments, the modified stem cell may include a human embryonic stem cell.

Some embodiments relate to a method for generating modified stem cells for transplantation. In some embodiments, the method may include culturing in a culture media the modified cells of the embodiments described above.

Some embodiments relate to a method for reducing inflammatory responses in response to transplantation. In some embodiments, the method may include culturing in a culture media the modified cells of the embodiments described above and administering a subject a composition including the modified cells.

In some embodiments, the transplantation is an allogeneic transplantation.

In some embodiments, accumulation of T lymphocytes in the subject in response to the transplantation is reduced as compared to transplantation of wild-type cells.

Some embodiments relate to a method for improving transplantation of stem cells or cells derived from the stem cells. In some embodiments, the method may include culturing in a culture media the modified cells of the embodiments described above, and administering a subject a composition comprising the modified cells. For example, immune responses of the subject in response to the transplantation is less than transplantation using wild type stem cells. The subject is an animal or a human.

In some embodiments, the transplantation is an allogeneic transplantation.

In some embodiments, the immune responses comprise accumulation of T lymphocytes in the subject in response to the transplantation.

In some embodiments, the immune responses comprise accumulation of NK lymphocytes in the subject in response to the transplantation.

In some embodiments, accumulation of NK lymphocytes in the subject response to the transplantation is reduced as compared to transplantation of wild-type cells. For example, the modified cells may be used in hematopoietic stem cell transplantation (HSCT). Under conventional techniques, the recipient's immune system has to be destroyed with radiation or chemotherapy before the transplantation. However, such pretreatment of radiation or chemotherapy may cause complications such as Infection and graft-versus-host disease. The recipient may die from these complications. The embodiments of the present disclosure may enable hematopoietic stem cell transplantation, especially in allogeneic HSCT, without destroying the recipient's immune system.

In some embodiments, the modified cells are transplanted into mice, both T and NK lymphocytes may be decreased in the injected sites compared to wild-type cells. As MHC class I can serve as a ligand of NK cells, the deficiency of MHC class I may lead to the decrease of NK cells in immune rejection. The expression deficiency of donor MHC class I may further cause failure to initiate the receptor's MHC class I recognition and the stimulation of T lymphocytes. Accordingly, TAP1− or TAPBP-deficient cell lines may be applied in transplantation therapy as a universal donor cell source.

The various embodiments described above can be combined to provide further embodiments. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

### EXAMPLE

### Generating TAP1− or TAPBP-Deficient hESC Lines

The hESCs were maintained in defined, feeder-free hESC and iPSC culture medium (mTeSR™ 1; STEMCELL Technologies) on matrigel-coated culture flasks without feeder cells. The feeder-free hESCs were passaged at a ratio of 1:8 every seven days. Dispase was used to dissociate the cells into cell clumps. Fresh matrigel-coated flasks should be prepared 20 min before cell passage. After about 30 min of dispase digestion, the hESCs were washed twice with hESC culture medium; meanwhile, the cell clumps should be dissociated into proper size by pipette before being passaged into new matrigel-coated culture flasks. The cryopreservation of TAP1− and TAPBP-deficient hESC colonies is necessary. Cryopreservation media (10% DMSO, 20% FBS, and 70% hESC culture medium) should be prepared in advance. The colonies' disassociation protocol was as the same as cell passage; then, the cells were transferred into cryopreservation media and allocated into CryoTube vials. The vials were put in cryocontainers and transferred into −80° C. refrigerator for temporary preservation. Cryopreserved cells need to be transferred into liquid nitrogen within 24 h. To form embryoid bodies, the hESCs were dissociated with dispase, as same as a passaging process, into smaller cell clumps. Cell clumps were then transferred onto a petri dish in DMEM (Invitrogen) supplemented with 20% FBS (Hyclone) for about six to nine days.

293T cells were maintained in DMEM (Invitrogen) supplemented with 10% FBS (Hyclone). 293T cells were split with trypsinization into single cells and passaged at a ratio of 1:8 every three days.

Firstly, it was confirmed that the TALENs-binding site of TAP1 or TAPBP gene, respectively, in hESC lines was identical with that in 293T cells by sequencing; so, instead of hESCs, 293T cells were used in a rapid TALENs efficiency evaluation for their high transfection efficiency feature. The TALENs-binding site was designed at the first exon for TAP1 and the second exon for TAPBP to make a frame shift mutation, resulting in generating the heterozygous or homozygous genotypes of hESCs.

The FastTALE TALEN Assembly Kit (Sidansai, Shanghai, China) was used to construct TALENs. The constructed TALENs were transfected into 293T cells using the FuGENE HD transfection reagent (Promega). Three days after transfection, the genomic DNA of 293T cells was harvested; direct PCR (F140, Life Technologies) and sequencing were performed to detect the efficiency of the TALENs. In general, 30 clones were examined at one time. But if the efficiency is low, another 30 clones were examined. The most efficient pairs were selected for the generation of gene-modified hESC lines.

The most efficient TALENs were co-transfected with puromycin resistance plasmid into feeder-free cultured hESCs using the FuGENE HD transfection reagent. The transfected colonies were screened by puromycin at a final concentration of 0.5 μg/mL three days after transfection. Colonies were dissociated into single cells using TrypLE (Invitrogen) and seeded onto newly pre-pared feeder (CF1) cells-coated plates at a density of 500 cells/cm2. Irradiated CF1 feeder cells should be seeded onto matrigel-coated culture flasks at a density of 30,000 cells/cm2 two days in advance. A single cell-derived colonies were transferred into two paralleled fresh CF1 feeder-coated wells for the establishment and identification of the mutants, respectively. The mutants were identified using a direct cell PCR kit (F140, Life Technologies) two weeks after passaging.

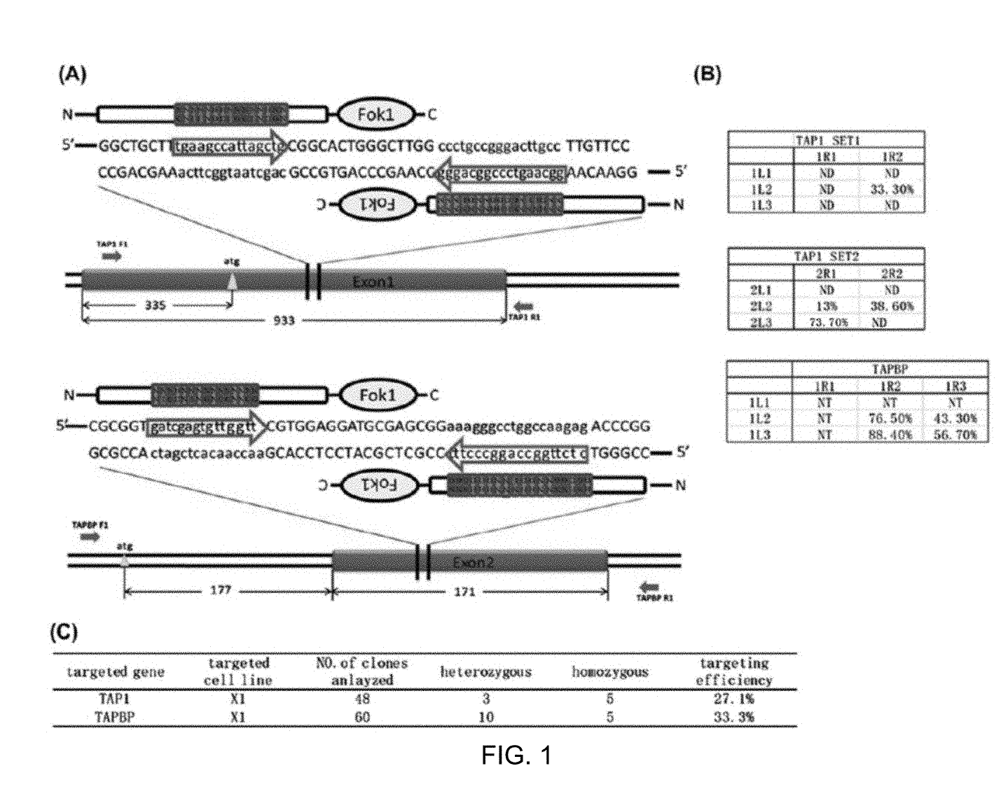

TALENs were used to disrupt TAP1 or TAPBP gene (FIG. 1(A)). Twelve pairs of TAP1− exonl-targeting TALENs and 9 pairs of TAPBP-exon2-targeting TALENs were constructed (FIG. 5A), respectively, and their activity was evaluated in 293T cells (FIG. 1(B)). TAP1− or TAPBP-deficient cell lines derived from hESC lines27 were constructed by the gene-targeting deletion of the most efficient TALEN pairs (2L3*2R1 for TAP1; 1L2*1R2 for TAPBP). Under the drug selection of puromycin, 27.1 and 33.3% clones were targeted in one or two alleles of TAP1 and TAPBP genes, respectively (FIG.1(C)). Heterozygous (TAP1+/- & TAPBP+/−) and homozygous (TAP1−/− & TAPBP−/−) hESCs (FIG. 5B) were used for further experiments (i.e., TAP1 X1-13, TAP1 X1-16, and TAP1 X1-29 as well as TAPBP X1-13 and TAPBP X1-11).

Flow Cytometry Analysis and Immunological Rejection Analysis.

HESCs were dissociated into single cells using TrypLE; then, cells were filtered with cell strainer, sand washed with phosphate buffered saline (PBS) supplemented with 2% FBS. Approximately, 5×105 cells were incubated in100 μL PBS supplemented with 2% FBS and PE-CY7-conjugated antibody for 30 min at 37° C. The primary antibody of HLA class I A, B, C (BD Biosciences) was used. FACS analysis was performed on a FACS Calibur flow cytometer system, equipped with FlowJo software (Tree Star. Ashland. Oreg.).

Approximately, 1×106 hESCs were resuspended in 100 μL culture medium and injected into the tibialis anterior muscles of three-four-week-old Balb/c mice. The muscles were harvested 48 h after cell transplantation and were fixed in 4% paraformaldehyde overnight, and then dehydrated in 20 and 30% sucrose solutions consecutively at 4° C. overnight. Then, the muscles were continuously sliced every 14 μm using a freezing microtome (Cryo-stat, Leica, Germany) and every 10 serial sections were selected for immunostaining. The primary antibodies of rabbit antibodies against mouse CD3 (1:300, Abcam) and mouse KLRA1 (1:500, Abcam) were used.

Statistical analysis was performed based on the CD3- or KLRA1-positive cells to detect the immune response in xenotransplantation.

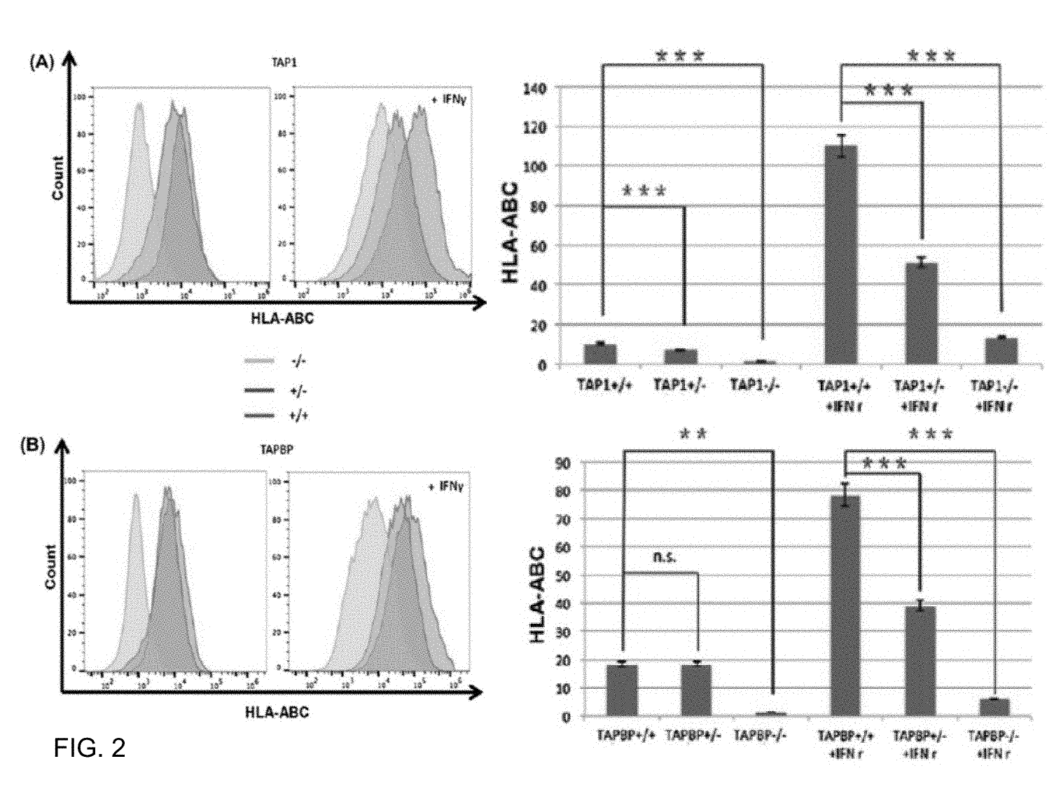

The FACS analysis of HLA-ABC for wild type, heterozygous (TAP1+/− & TAPBP+/−), and homozygous (TAP1−/− &TAPBP−/−) hESCs showed that the expression level of MHC class I was markedly reduced in either TAP1− or TAPBP-deficient cell lines, especially in TAP1−/− and TAPBP−/− cell lines (FIG. 2). Interferon gamma (IFN-γ) has been reported to be able to up-regulate MHC class I expression, so these cell lines were treated with IFN-γ for 24 hs, and then FACS analysis was performed. Even IFN-y treatment cannot res-cue the expression of MHC class I significantly (FIG. 2). Interestingly, it was found that the expression levels of HLA-ABC were up-regulated even in the TAP-1 or TAP-BP knock-out cells by the IFN treatment. Accordingly, HLA-ABC peptides can be transported by TAP-2 or there is any compensating material that may compensate for the loss of TAP-1 or TAP-BP.

Reverse Transcription and Real-Time PCR and Cell Immunostaining

The whole RNA was prepared using an RNeasy kit (Qiagen). RevertAid Reverse Transcriptase (Thermo) was used for reverse transcribing the RNA into cDNA which was then used as a template for real-time polymerase chain reaction (RT-PCR). RT-PCR was operated in an Eppendorf Mastercycler® ep realplex real-time PCR system using SYBR Green-based PCR Master Mix (TOYOBO). Standard curves were acquired with an internal control (G3PD). The primers for RT-PCR are listed in Supplementary Table 1.

Immunostaining was carried out similarly as previously described.26) The primary antibodies of anti-Oct4 (1:100, Santa Cruz Biotechnology), anti-Nanog (1:150, Santa Cruz Biotechnology), anti-SSEA3 (1:400, Developmental Studies Hybridoma Bank), and anti-Tra-1-60 (1:150, Chemicon) were used. Alexa Flour® 594 donkey anti-goat IgG (H +L) secondary antibodies were used for Oct4 and Nanog, Alexa Flour® 594 goat anti-rat IgM (μ chain) secondary antibodies for SSEA3, and Alexa Flour® 546 goat anti-mouse IgM (μ chain) secondary antibodies for Tra-1-60. Nuclei were stained with DAPI as counterstaining. The feeder-free-cultured hESC colonies were washed with PBS twice and then fixed in 4% paraformaldehyde for 30 min at room temperature. For nuclear proteins, such as Oct4 and Nanog, cells were permeabilized in 100% ethanol thrice (10mins each). After permeabilization, the cells were washed with PBS once, in the antibody dilution buffer (PBS with 0.2% BSA and 0.1% TritonX-100, 5 min each) twice, and then blocked in a blocking solution (PBS with 1% BSA, 4% normal serum, and 0.4% TritonX-100) for 1 h at room temperature. The cells were incubated with the primary antibodies overnight at 4° C. Then, the cells were washed with PBS containing 0.1% TritonX-100 (PBS-T, 5 min each) thrice and incubated with the

Secondary antibodies for 1 h at 37° C. in the dark. The cells were then washed in PBS-T thrice and incubated with Teratoma formation. Approximately, 5×106 hESCs were resuspended in 200-μL culture medium and injected into the hind leg of six-eight-week-old non-obese diabetic/severe combined immune-deficient (NOD/SCID) mice intramuscularly. After 6-8 weeks, tumors were harvested, fixed in 4% paraformaldehyde, and examined histologically the following hematoxylin—eosin (HE) staining. All animal experiments complied with the Guide for the Care and Use of Animals for Research Purposes and were approved by the Zhejiang University Animal Care Committee.

Data were represented as the mean±SEM. Statistical significance was calculated using an unpaired Dunnett test, and differences were considered significant at p<005.

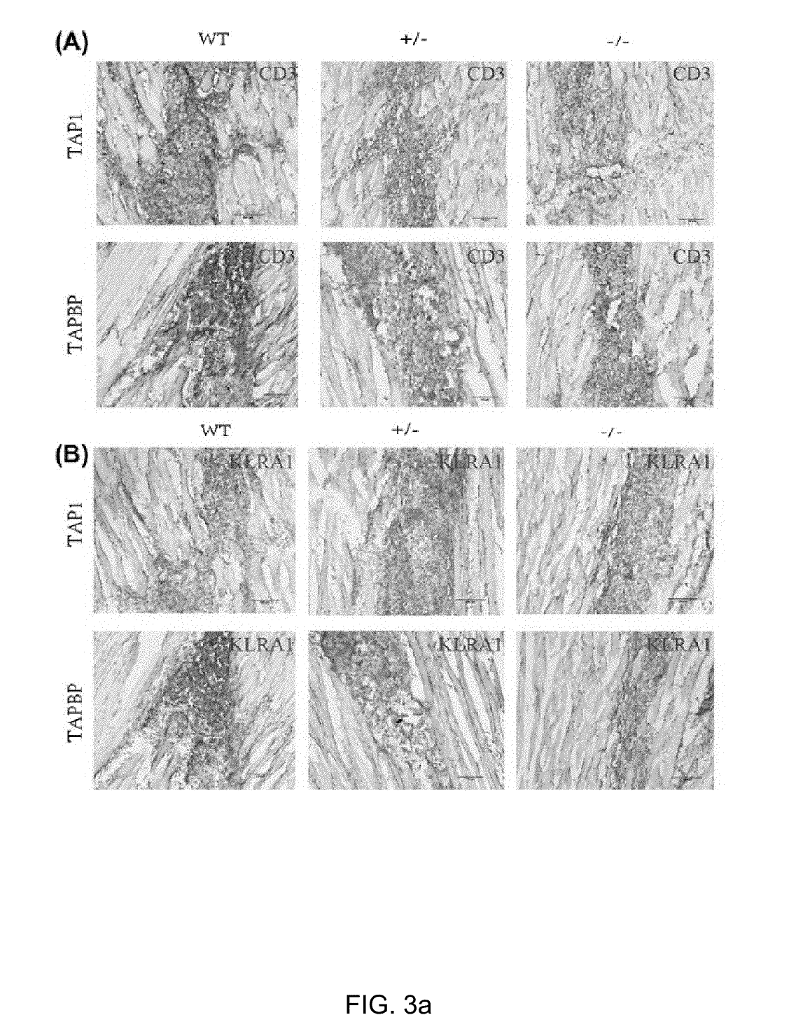

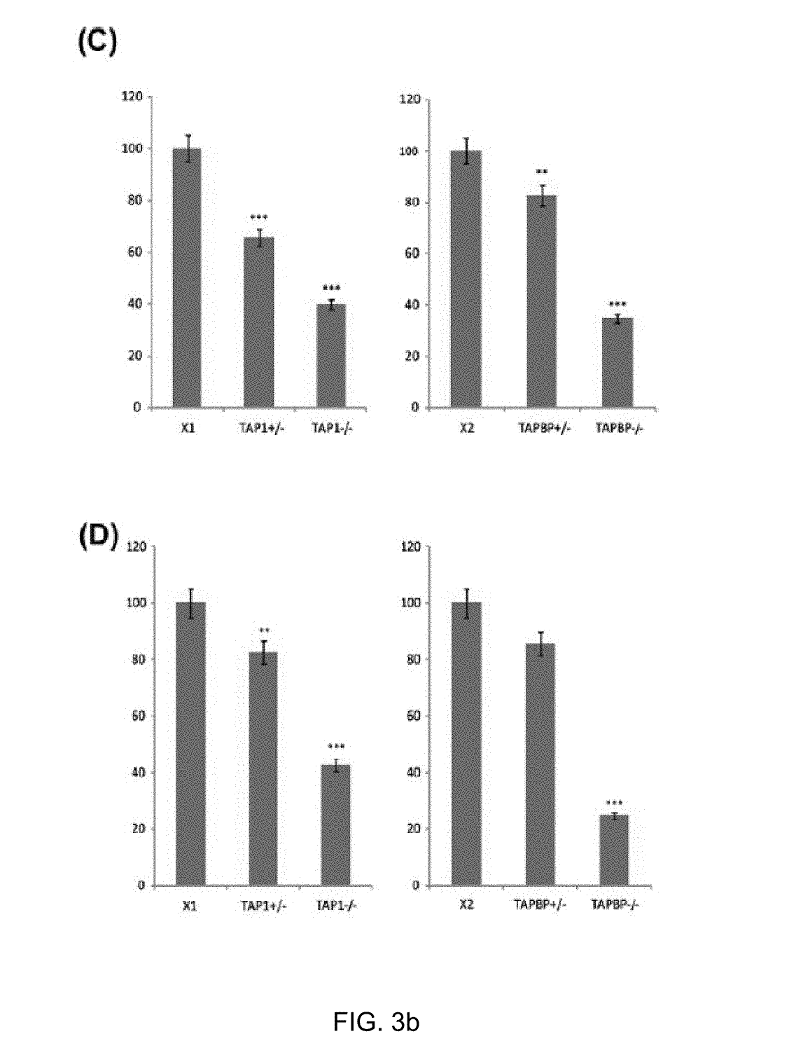

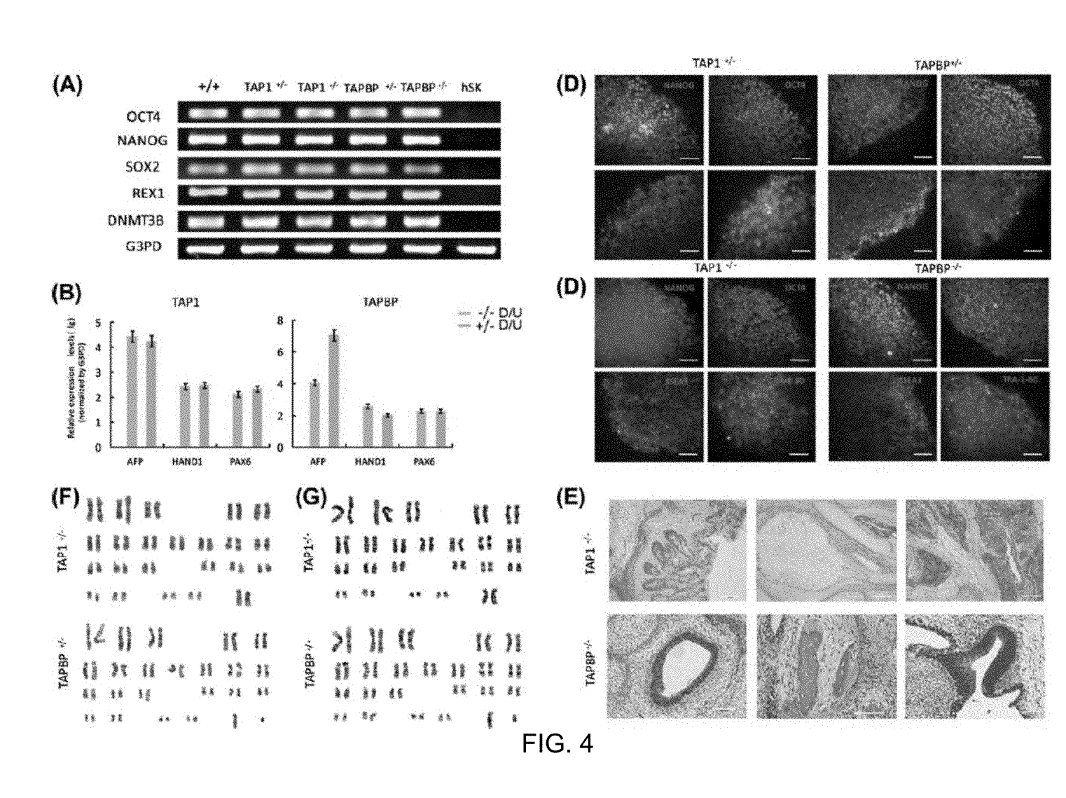

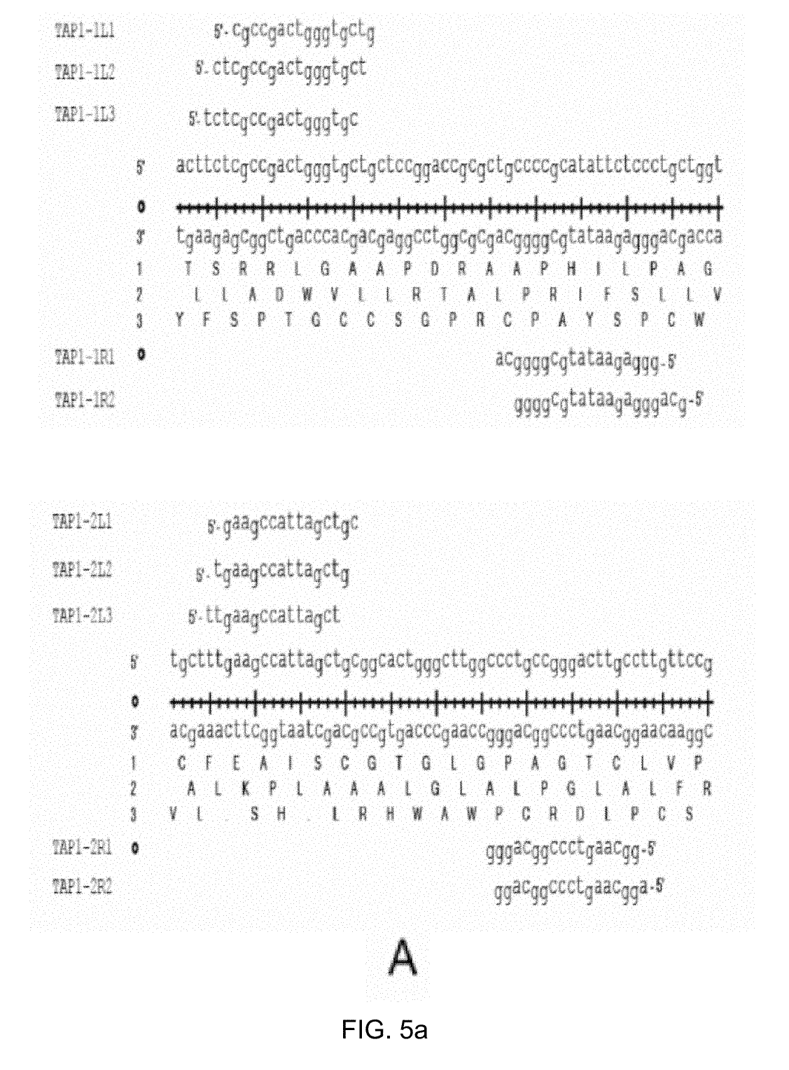

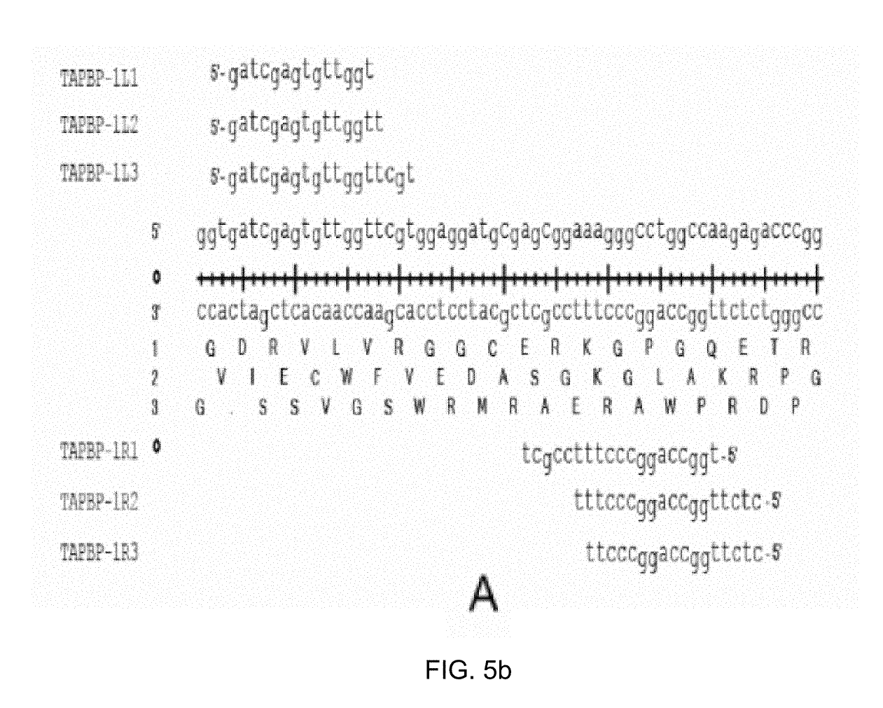

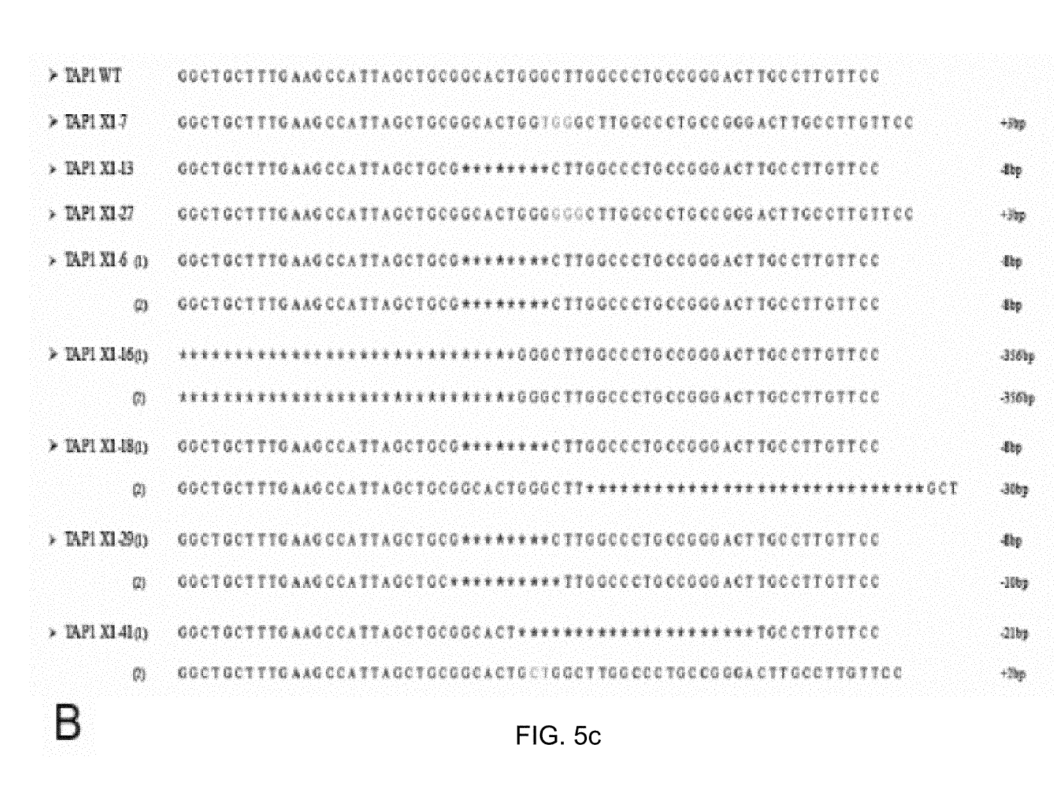

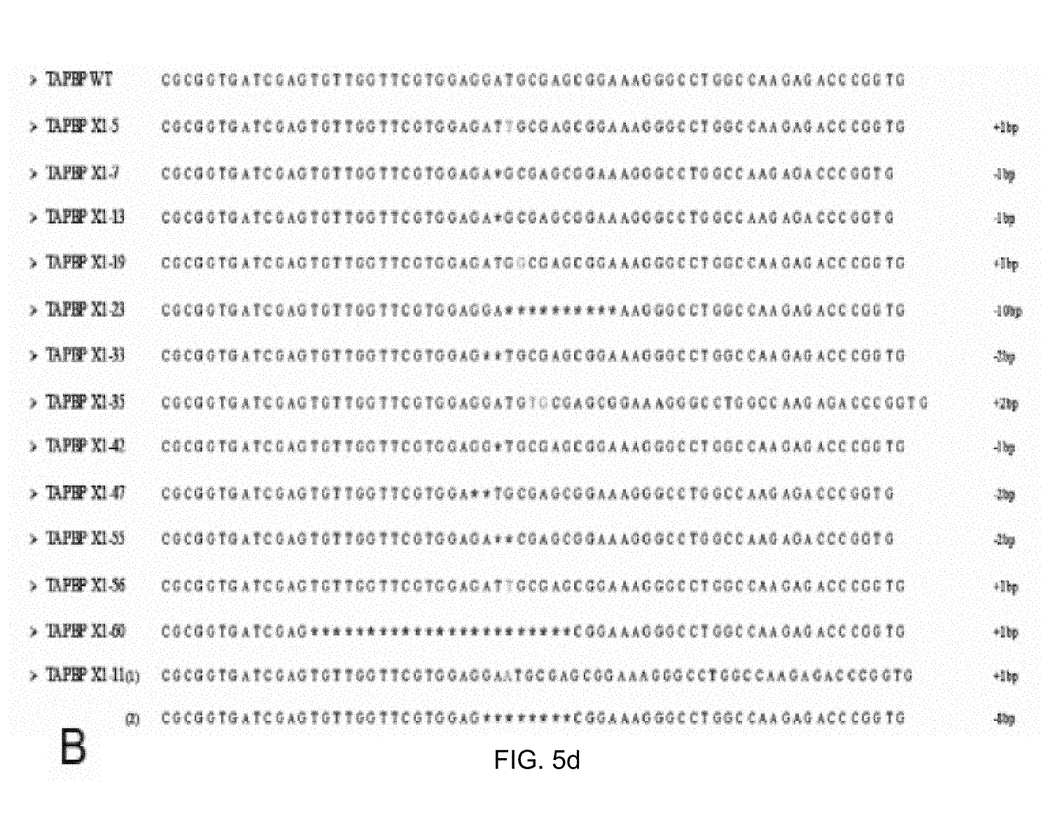

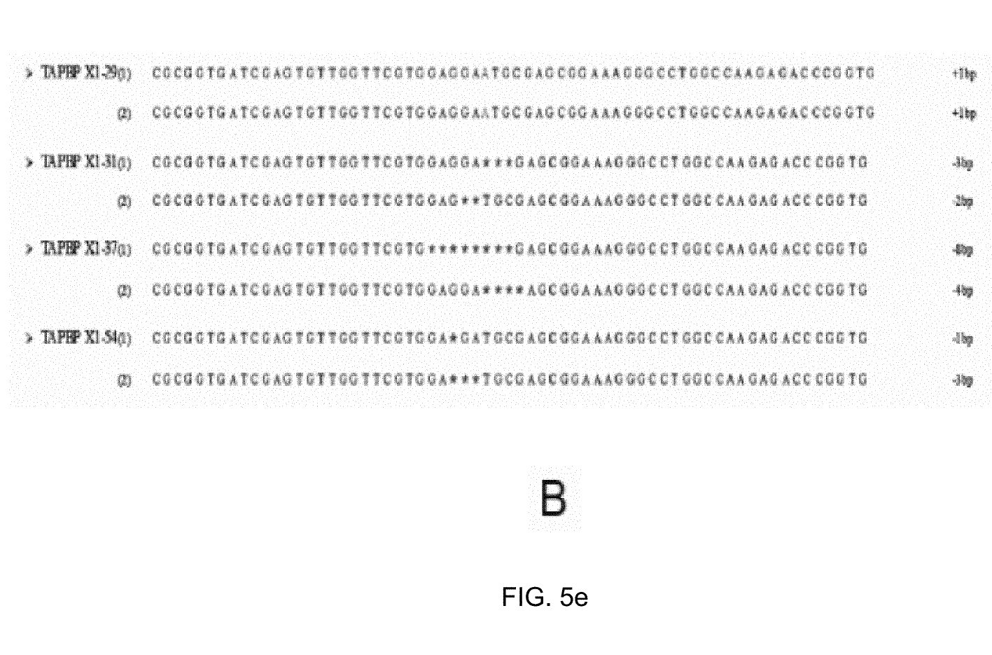

To investigate the immune rejection of mutant hESCs in cell transplantation, wild type, heterozygous (TAP1+/− & TAPBP+/−), and homozygous (TAP1−/− & TAPBP−/−) hESCs were injected into tibialis anterior muscles of Balb/c mice. The immunostaining of the tibialis anterior muscles harvested 48 h post-injection showed that both T lymphocytes (FIG. 3(A) and (C)) and NK lymphocytes (FIG. 3(B) and (D)) were less accumulated for TAP1− or TAPBP-deficient hESCs in comparison to wild type.

The pluripotency of both heterozygous and homozygous hESCs lines was detected by real-time PCR, immunostaining, and teratoma formation analysis.

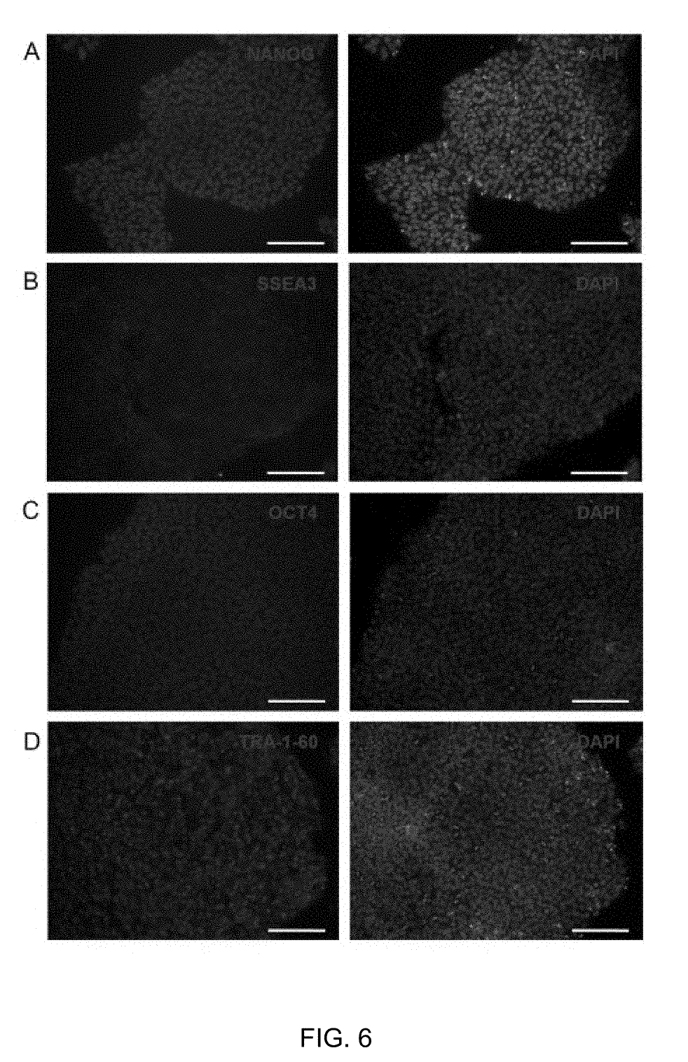

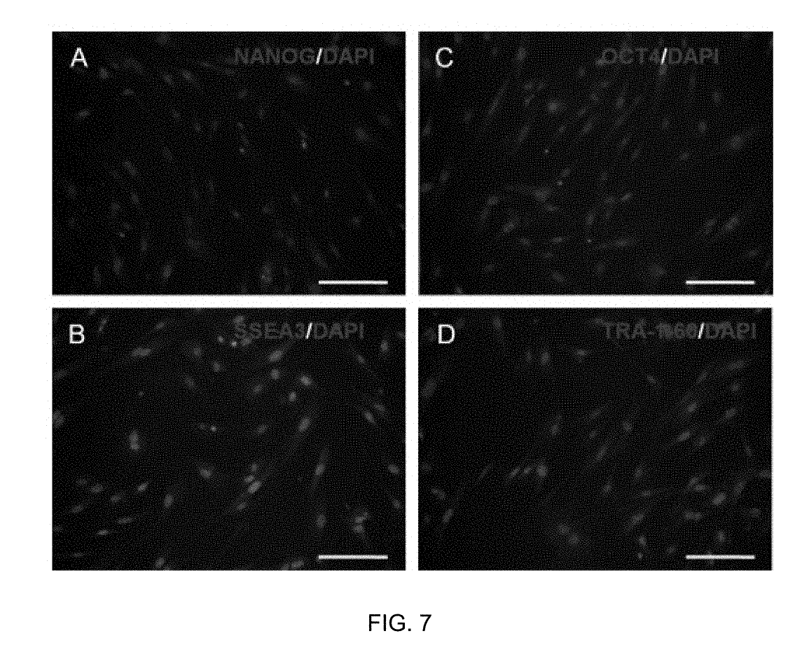

Reverse transcription showed no differences between wild types and TAP1− or TAPBP-deficient cell lines in pluripotent genes, including Oct4, Nanog, Sox2, Nodal, Dnmt3b, and Rex1 (FIG. 4(A)). Embryoid bodies derived from TAP1− or TAPBP-deficient hESC lines expressed the marker genes of three germ layers, such as AFP, HAND, and PAX6 (FIG. 4(B)).

Immunostaining showed that heterozygous, homozygous TAP1− or TAPBP-deficient cell and lines expressed pluripotent proteins, including Oct4, Nanog, SSEA3, and Tra-1-60 as wild types (FIG. 4(C) and (D), FIGS. 6 and 7).

Teratomas could be formed two months post-injection of heterozygous and homozygous TAP1− or TAPBP-deficient hESCs lines, and tissues from all three germ layers were seen in HE staining sections of the teratomas (FIG. 4(E)). Besides, both TAP (FIG. 4(F)) and TAPBP (FIG. 4(G)) hESC lines demonstrated normal karyotypes.

Various sequences described above are listed in Tables 1, 2, and 3 as follow.

TABLE 1

| SEQ | | |
|---|---|---|
| ID NO: | Identifier | Sequence |
| 1 | Primer Oct4-F | GACAGGGGGAGGGGAGGAGCTAGG |
| 2 | Primer Oct4-R | CTTCCCTCCAACCAGTTGCCCCAA |
| | | AC |
| 3 | Primer Sox2-F | GGGAAATGGGAGGGGTGCAAAAGA |
| | | GG |
| 4 | Primer Sox2-R | TTGCGTGAGTGTGGATGGGATTGG |
| | | TG |
| 5 | Primer Nanog-F | CAGCCCCGATTCTTCCACCAGTCC |
| | | C |
| 6 | Primer Nanog-R | CGGAAGATTCCCAGTCGGGTTCAC |
| | | C |
| 7 | Primer Rex1-F | CAGATCCTAAACAGCTCGCAGAAT |
| 8 | Primer Rex1-R | GCGTACGCAAATTAAAGTCCAGA |
| 9 | Primer Dnmt3b-F | TGCTGCTCACAGGGCCCGATACTT |
| | | C |
| 10 | Primer Dnmt3b-R | TCCTTTCGAGCTCAGTGCACCACA |
| | | AAAC |
| 11 | Primer Nodal-F | GGGCAAGAGGCACCGTCGACATCA |
| 12 | Primer Nodal-R | GGGACTCGGTGGGGCTGGTAACGT |
| | | TTC |
| 13 | Primer Afp-F | GAATGCTGCAAACTGACCACGCTG |
| | | GAAC |
| 14 | Primer Afp-R | TGGCATTCAAGAGGGTTTTCAGTC |
| | | TGGA |
| 15 | Primer Hand1-F | TGCCTGAGAAAGAGAACCAG |
| 16 | Primer Hand1-R | ATGGCAGGATGAACAAACAC |
| 17 | Primer Pax6-F | TACCAACCAATTCCACAACCCACC |
| 18 | Primer Pax6-R | ATCATAACTCCGCCCATTCACCG |
| 19 | Primer G3pd-F | AGGTCGGAGTCAACGGATTTGG |
| 20 | Primer G3pd-R | AGGCTGTTGTCATACTTCTCATGG |
| 21 | Target TAP1 | GGCTGCTTTGAAGCCATTAGCTGC |
| | Exon 1 | GGCACTGGGCTTGGCCCTGCCGGG |
| | | ACTTGCCTTGTTCC |
| 22 | Target TAP1 | GGAACAAGGCAAGTCCCGGCAGGG |
| | Exon1 | CCAAGCCCAGTGCCGCAGCTAATG |
| | | GCTTCTAAAGCAGCC |
| 23 | Left arm of | TGAAGCCATTAGCTG |
| | TAP1 deletion | |
| 24 | Right arm of | GGCAAGTCCCGGCAGGG |
| | TAP1 deletion | |
| 25 | Target TAPBP | CGCGGTGATCGAGTGTTGGTTCGT |
| | Exon 2 | GGAGGATGCGAGCGGAAAGGGCCT |
| | | GGCCAAGAGACCCGG |
| 26 | Target TAPBP | CCGGGTCTCTTGGCCAGGCCCTTT |
| | Exon 2 | CCGCTCGCATCCTCCACGAACCAA |
| | | CACTCGATCACCGCG |
| 27 | Left arm of | GATCGAGTGTTGGTT |
| | TAPBP | |
| | deflection | |
| 28 | Right arm of | CTCTTGGCCAGGCCCTTT |
| | TAPBP | |
| | deflection | |
| 29 | TAP1 exon1 | GTGTGCGTGATGGAGAAAATTGGG |
| | | CACCAGGGCTGCTCCCGAGATTCT |
| | | CAGATCTGATTTCCACGCTTGCTA |
| | | CCAAAATAGTCTGGGCAGGCCACT |
| | | TTTGGAAGTAGGCGTTATCTAGTG |
| | | AGCAGGCGGCCGCTTTCGATTTCG |
| | | CTTTCCCCTAAATGGCTGAGCTTC |
| | | TCGCCAGCGCAGGATCAGCCTGTT |
| | | CCTGGGACTTTCCGAGAGCCCCGC |
| | | CCTCGTTCCCTCCCCCAGCCGCCA |
| | | GTAGGGGAGGACTCGGCGGTACCC |
| | | GGAGCTTCAGGCCCCACCGGGGCG |
| | | CGGAGAGTCCCAGGCCCGGCCGGG |
| | | ACCGGGACGGCGTCCGAGTGCCAA |
| | | TGGCTAGCTCTAGGTGTCCCGCTC |
| | | CCCGCGGGTGCCGCTGCCTCCCCG |
| | | GAGCTTCTCTCGCATGGCTGGGGA |
| | | CAGTACTGCTACTTCTCGCCGACT |
| | | GGGTGCTGCTCCGGACCGCGCTGC |
| | | CCCGCATATTCTCCCTGCTGGTGC |
| | | CCACCGCGCTGCCACTGCTCCGGG |
| | | TCTGGGCGGTGGGCCTGAGCCGCT |
| | | GGGCCGTGCTCTGGCTGGGGGCCT |
| | | GCGGGGTCCTCAGGGCAACGGTTG |
| | | GCTCCAAGAGCGAAAACGCAGGTG |
| | | CCCAGGGCTGGCTGGCTGCTTTGA |
| | | AGCCATTAGCTGCGGCACTGGGCT |
| | | TGGCCCTGCCGGGACTTGCCTTGT |
| | | TCCGAGAGCTGATCTCATGGGGAG |
| | | CCCCCGGGTCCGCGGATAGCACCA |
| | | GGCTACTGCACTGGGGAAGTCACC |
| | | CTACCGCCTTCGTTGTCAGTTATG |
| | | CAGCGGCACTGCCCGCAGCAGCCC |
| | | TGTGGCACAAACTCGGGAGCCTCT |
| | | GGGTGCCCGGCGGTCAGGGCGGCT |
| | | CTGGAAACCCTGTGCGTCGGCTTC |
| | | TAGGCTGCCTGGGCTCGGAGACGC |
| | | GCCGCCTCTCGCTGTTCCTGGTCC |
| | | TGGTGGTCCTCTCCTCTCTTG |
| 30 | TAPBP exon 2 | GCCTGGCGACCGCCGTCTCAGCAG |
| | | GACCCGCGGTGATCGAGTGTTGGT |
| | | TCGTGGAGGATGCGAGCGGAAAGG |
| | | GCCTGGCCAAGAGACCCGGTGCAC |
| | | T81GCTGTTGCGCCAGGGACCGGG |
| | | GGAACCGCCGCCCCGGCCGGACCT |
| | | CGACCCTGAGCTCTA541TCTCAG |
| | | TGTACACGACCCCGCGGG |

TABLE 2
TAP1 knockout cell lines affected
amino acid sequence from ID 35)

| SEQ | | |
|---|---|---|
| ID NO: | Identifier | Sequence |
| 21 | Target | G G C T G C T T T G A A G C C A T T A G C T G |
| | TAP1 WT | C G G C A C T G G G C T T G G C C C T G C C G |
| | | G G A C T T G C C T T G T T C C |
| 31 | TAP1 X1-7 | G G C T G C T T T G A A G C C A T T A G C T G |
| | | C G G C A C T G G T G G G C T T G G C C C T G |
| | | C C G G G A C T T G C C T T G T T C C |
| 32 | TAP1 X1-13 | G G C T G C T T T G A A G C C A T T A G C T G |
| | | C G C T T G G C C C T G C C G G G A C T T G C |
| | | C T T G T T C C |
| 33 | TAP1 X1-27 | G G C T G C T T T G A A G C C A T T A G C T G |
| | | C G G C A C T G G G G G G C T T G G C C C T G |
| | | C C G G G A C T T G C C T T G T T C C |
| 34 | TAP1 X1-6 | G G C T G C T T T G A A G C C A T T A G C T G |
| | (1) | C G C T T G G C C C T G C C G G G A C T T G C |
| | | C T T G T T C C |
| 35 | (2) | G G C T G C T T T G A A G C C A T T A G C T G |
| | | C G C T T G G C C C T G C C G G G A C T T G C |
| | | C T T G T T C C |
| 36 | TAP1 X1-16 | G G G C T T G G C C C T G C C G G G A C T T G |
| | (1) | C C T T G T T C C |
| 37 | (2) | G G G C T T G G C C C T G C C G G G A C T T G |
| | | C C T T G T T C C |
| 38 | TAP1 X1-16 | G G C T G C T T T G A A G C C A T T A G C T G |
| | deletion | C G G C A C T |
| 39 | TAP1 X1-18 | G G C T G C T T T G A A G C C A T T A G C T G |
| | (1) | C G C T T G G C C C T G C C G G G A C T T G C |
| | | C T T G T T C C |
| 40 | (2) | G G C T G C T T T G A A G C C A T T A G C T G |
| | | C G G C A C T G G G C T T G C T |
| 41 | TAP1 X1-29 | G G C T G C T T T G A A G C C A T T A G C T G |
| | (1) | C G C T T G G C C C T G C C G G G A C T T G C |
| | | C T T G T T C C |
| 42 | (2) | G G C T G C T T T G A A G C C A T T A G C T G |
| | | C T T G G C C C T G C C G G G A C T T G C C T |
| | | T G T T C C |
| 43 | TAP1 X1- | G G C T G C T T T G A A G C C A T T A G C T G |
| | 41(1) | C G G C A C T T G C C T T G T T C C |
| 44 | (2) | G G C T G C T T T G A A G C C A T T A G C T G |
| | | C G G C A C T G C T G G C T T G G C C C T G C |
| | | C G G G A C T T G C C T T G T T C C |

TABLE 3
TAPBP knockout cell lines
(affected amino acid sequence from ID 27)

| SEQ | | |
|---|---|---|
| ID NO: | Identifier | Sequence |
| 25 | Target | C G C G G T G A T C G A G T G T T G G T T C G |
| | TAPBP WT | T G G A G G A T G C G A G C G G A A A G G G C |
| | | C T G G C C A A G A G A C C C G G T G |
| 45 | TAPBPX1-5 | C G C G G T G A T C G A G T G T T G G T T C G |
| | | T G G A G A T T G C G A G C G G A A A G G G C |
| | | C T G G C C A A G A G A C C C G G T G |
| 46 | TAPBPX1-7 | C G C G G T G A T C G A G T G T T G G T T C G |
| | | T G G A G A G C G A G C G G A A A G G G C C T |
| | | G G C C A A G A G A C C C G G T G |
| 47 | TAPBP X1- | C G C G G T G A T C G A G T G T T G G T T C G |
| | 13 | T G G A G A G C G A G C G G A A A G G G C C T |
| | | G G C C A A G A G A C C C G G T G |
| 48 | TAPBP X1- | C G C G G T G A T C G A G T G T T G G T T C G |
| | 19 | T G G A G A T G G C G A G C G G A A A G G G C |
| | | C T G G C C A A G A G A C C C G G T G |
| 49 | TAPBP X1- | C G C G G T G A T C G A G T G T T G G T T C G |
| | 23 | T G G A G G A A A G G G C C T G G C C A A G A |
| | | G A C C C G G T G |
| 50 | TAPBP X1- | C G C G G T G A T C G A G T G T T G G T T C G |
| | 33 | T G G A G T G C G A G C G G A A A G G G C C T |
| | | G G C C A A G A G A C C C G G T G |
| 51 | TAPBP X1- | C G C G G T G A T C G A G T G T T G G T T C G |
| | 35 | T G G A G G A T G T G C G A G C G G A A A G G |
| | | G C C T G G C C A A G A G A C C C G G T G |
| 52 | TAPBP X1- | C G C G G T G A T C G A G T G T T G G T T C G |
| | 42 | T G G A G G T G C G A G C G G A A A G G G C C |
| | | T G G C C A A G A G A C C C G G T G |
| 53 | TAPBP X1- | C G C G G T G A T C G A G T G T T G G T T C G |
| | 47 | T G G A T G C G A G C G G A A A G G G C C T G |
| | | G C C A A G A G A C C C G G T G |
| 54 | TAPBP X1- | C G C G G T G A T C G A G T G T T G G T T C G |
| | 55 | T G G A G A C G A G C G G A A A G G G C C T G |
| | | G C C A A G A G A C C C G G T G |
| 55 | TAPBP X1- | C G C G G T G A T C G A G T G T T G G T T C G |
| | 56 | T G G A G A T T G C G A G C G G A A A G G G C |
| | | C T G G C C A A G A G A C C C G G T G |
| 56 | TAPBP X1- | C G C G G T G A T C G A G C G G A A A G G G C |
| | 60 | C T G G C C A A G A G A C C C G G T G |
| 57 | TAPBP X1- | C G C G G T G A T C G A G T G T T G G T T C G |
| | 11 | T G G A G G A A T G C G A G C G G A A A G G G |
| | (1) | C C T G G C C A A G A G A C C C G G T G |
| 58 | (2) | C G C G G T G A T C G A G T G T T G G T T C G |
| | | T G G A G C G G A A A G G G C C T G G C C A A |
| | | G A G A C C C G G T G |
| 59 | TAPBP X1- | C G C G G T G A T C G A G T G T T G G T T C G |
| | 29 | T G G A G G A A T G C G A G C G G A A A G G G |
| | (1) | C C T G G C C A A G A G A C C C G G T G |
| 60 | (2) | C G C G G T G A T C G A G T G T T G G T T C G |
| | | T G G A G G A A T G C G A G C G G A A A G G G |
| | | C C T G G C C A A G A G A C C C G G T G |
| 61 | TAPBP X1- | C G C G G T G A T C G A G T G T T G G T T C G |
| | 31 | T G G A G G A G A G C G G A A A G G G C C T G |
| | (1) | G C C A A G A G A C C C G G T G |
| 62 | (2) | C G C G G T G A T C G A G T G T T G G T T C G |
| | | T G G A G T G C G A G C G G A A A G G G C C T |
| | | G G C C A A G A G A C C C G G T G |
| 63 | TAPBP X1- | C G C G G T G A T C G A G T G T T G G T T C G |
| | 37 | T G G A G C G G A A A G G G C C T G G C C A A |
| | (1) | G A G A C C C G G T G |
| 64 | (2) | C G C G G T G A T C G A G T G T T G G T T C G |
| | | T G G A G G A A G C G G A A A G G G C C T G G |
| | | C C A A G A G A C C C G G T G |
| 65 | TAPBP X1- | C G C G G T G A T C G A G T G T T G G T T C G |
| | 54 | T G G A G A T G C G A G C G G A A A G G G C C |
| | (1) | T G G C C A A G A G A C C C G G T G |
| 66 | (2) | C G C G G T G A T C G A G T G T T G G T T C G |
| | | T G G A T G C G A G C G G A A A G G G C C T G |
| | | G C C A A G A G A C C C G G T G |

TABLE 4
TAP1 knockout cell lines

| SEQ ID | | |
|---|---|---|
| NO: | Identifier | Sequence |
| 29 | TAP1 WT | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | GGCTGCTTTGAAGCCATTAGCTGCGGCACTGGGCTTGGCCCTGC |
| | | CGGGACTTGCCTTGTTCCGAGAGCTGATCTCATGGGGAGCCCCC |
| | | GGGTCCGCGGATAGCACCAGGCTACTGCACTGGGGAAGTCACC |
| | | CTACCGCCTTCGTTGTCAGTTATGCAGCGGCACTGCCCGCAGCA |
| | | GCCCTGTGGCACAAACTCGGGAGCCTCTGGGTGCCCGGCGGTC |
| | | AGGGCGGCTCTGGAAACCCTGTGCGTCGGCTTCTAGGCTGCCTG |
| | | GGCTCGGAGACGCGCCGCCTCTCGCTGTTCCTGGTCCTGGTGGT |
| | | CCTCTCCTCTCTTG |
| 67 | TAP1 X1-7 | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G G C A C T G G T G G G C T T G G C C C T G C C |
| | | G G G A C T T G C C T T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 68 | TAP1 X1-13 | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G C T T G G C C C T G C C G G G A C T T G C C T |
| | | T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 69 | TAP1 X1-27 | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G G C A C T G G G G G G C T T G G C C C T G C C |
| | | G G G A C T T G C C T T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 70 | TAP1 X1-6 | GTGTGCGTGATGGAGAAAATTGGGCACCAGGGCTGCTCCCGAG |
| | (1) | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G C T T G G C C C T G C C G G G A C T T G C C T |
| | | T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 71 | (2) | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G C T T G G C C C T G C C G G G A C T T G C C T |
| | | T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 72 | TAP1 X1-16 | GTGTGCGTGATGGAGAAAATTGGGCACCAGGGCTGCTCCCGAG |
| | (1) | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGGGCTTGGCCCTGCCGGGACT |
| | | TGCCTTGTTCCGAGAGCTGATCTCATGGGGAGCCCCCGGGTCCG |
| | | CGGATAGCACCAGGCTACTGCACTGGGGAAGTCACCCTACCGCC |
| | | TTCGTTGTCAGTTATGCAGCGGCACTGCCCGCAGCAGCCCTGTG |
| | | GCACAAACTCGGGAGCCTCTGGGTGCCCGGCGGTCAGGGCGGC |
| | | TCTGGAAACCCTGTGCGTCGGCTTCTAGGCTGCCTGGGCTCGGA |
| | | GACGCGCCGCCTCTCGCTGTTCCTGGTCCTGGTGGTCCTCTCCTC |
| | | TCTTG |
| 73 | (2) | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGGGCTTGGCCCTGCCGGGACT |
| | | TGCCTTGTTCCGAGAGCTGATCTCATGGGGAGCCCCCGGGTCCG |
| | | CGGATAGCACCAGGCTACTGCACTGGGGAAGTCACCCTACCGCC |
| | | TTCGTTGTCAGTTATGCAGCGGCACTGCCCGCAGCAGCCCTGTG |
| | | GCACAAACTCGGGAGCCTCTGGGTGCCCGGCGGTCAGGGCGGC |
| | | TCTGGAAACCCTGTGCGTCGGCTTCTAGGCTGCCTGGGCTCGGA |
| | | GACGCGCCGCCTCTCGCTGTTCCTGGTCCTGGTGGTCCTCTCCTC |
| | | TCTTG |
| 74 | TAP1 X1-16 | GCGGAGAGTCCCAGGCCCGGCCGGGACCGGGACGGCGTCCGA |
| | deletion | GTGCCAATGGCTAGCTCTAGGTGTCCCGCTCCCCGCGGGTGCCG |
| | | CTGCCTCCCCGGAGCTTCTCTCGCATGGCTGGGGACAGTACTGC |
| | | TACTTCTCGCCGACTGGGTGCTGCTCCGGACCGCGCTGCCCCGC |
| | | ATATTCTCCCTGCTGGTGCCCACCGCGCTGCCACTGCTCCGGGTC |
| | | TGGGCGGTGGGCCTGAGCCGCTGGGCCGTGCTCTGGCTGGGG |
| | | GCCTGCGGGGTCCTCAGGGCAACGGTTGGCTCCAAGAGCGAAA |
| | | ACGCAGGTGCCCAGGGCTGGCTGGCTGCTTTGAAGCCATTAGCT |
| | | GCGGCACT |
| 75 | TAP1 X1-18 | GTGTGCGTGATGGAGAAAATTGGGCACCAGGGCTGCTCCCGAG |
| | (1) | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G C T T G G C C C T G C C G G G A C T T G C C T |
| | | T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 76 | (2) | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G G C A C T G G G C T T G C T |
| | | GATCTCATGGGGAGCCCCCGGGTCCGCGGATAGCACCAGGCTAC |
| | | TGCACTGGGGAAGTCACCCTACCGCCTTCGTTGTCAGTTATGCAG |
| | | CGGCACTGCCCGCAGCAGCCCTGTGGCACAAACTCGGGAGCCT |
| | | CTGGGTGCCCGGCGGTCAGGGCGGCTCTGGAAACCCTGTGCGT |
| | | CGGCTTCTAGGCTGCCTGGGCTCGGAGACGCGCCGCCTCTCGCT |
| | | GTTCCTGGTCCTGGTGGTCCTCTCCTCTCTTG |
| 77 | TAP1 X1-29 | GTGTGCGTGATGGAGAAAATTGGGCACCAGGGCTGCTCCCGAG |
| | (1) | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G C T T G G C C C T G C C G G G A C T T G C C T |
| | | T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 78 | (2) | G G C T G C T T T G A A G C C A T T A G C T G C |
| | | T T G G C C C T G C C G G G A C T T G C C T T G |
| | | T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 79 | TAP1 X1-41 | GTGTGCGTGATGGAGAAAATTGGGCACCAGGGCTGCTCCCGAG |
| | (1) | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G G C A C T T G C C T T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |
| 80 | (2) | GTGTGCGTGATGGAGAAAATTGGGCACCAGGGCTGCTCCCGAG |
| | | ATTCTCAGATCTGATTTCCACGCTTGCTACCAAAATAGTCTGGGCA |
| | | GGCCACTTTTGGAAGTAGGCGTTATCTAGTGAGCAGGCGGCCGC |
| | | TTTCGATTTCGCTTTCCCCTAAATGGCTGAGCTTCTCGCCAGCGC |
| | | AGGATCAGCCTGTTCCTGGGACTTTCCGAGAGCCCCGCCCTCGT |
| | | TCCCTCCCCCAGCCGCCAGTAGGGGAGGACTCGGCGGTACCCG |
| | | GAGCTTCAGGCCCCACCGGGGCGCGGAGAGTCCCAGGCCCGG |
| | | CCGGGACCGGGACGGCGTCCGAGTGCCAATGGCTAGCTCTAGG |
| | | TGTCCCGCTCCCCGCGGGTGCCGCTGCCTCCCCGGAGCTTCTCTC |
| | | GCATGGCTGGGGACAGTACTGCTACTTCTCGCCGACTGGGTGCT |
| | | GCTCCGGACCGCGCTGCCCCGCATATTCTCCCTGCTGGTGCCCAC |
| | | CGCGCTGCCACTGCTCCGGGTCTGGGCGGTGGGCCTGAGCCGC |
| | | TGGGCCGTGCTCTGGCTGGGGGCCTGCGGGGTCCTCAGGGCAA |
| | | CGGTTGGCTCCAAGAGCGAAAACGCAGGTGCCCAGGGCTGGCT |
| | | G G C T G C T T T G A A G C C A T T A G C T G C |
| | | G G C A C T G C T G G C T T G G C C C T G C C G |
| | | G G A C T T G C C T T G T T C C |
| | | GAGAGCTGATCTCATGGGGAGCCCCCGGGTCCGCGGATAGCAC |
| | | CAGGCTACTGCACTGGGGAAGTCACCCTACCGCCTTCGTTGTCA |
| | | GTTATGCAGCGGCACTGCCCGCAGCAGCCCTGTGGCACAAACTC |
| | | GGGAGCCTCTGGGTGCCCGGCGGTCAGGGCGGCTCTGGAAACC |
| | | CTGTGCGTCGGCTTCTAGGCTGCCTGGGCTCGGAGACGCGCCGC |
| | | CTCTCGCTGTTCCTGGTCCTGGTGGTCCTCTCCTCTCTTG |

1. A modified cell comprising a reduced amount of Major Histocompatibility Complex I (MHC I) as compared to a corresponding wild-type cell, wherein the modified cell has decreased immunogenicity as compared to the corresponding wild-type cell, and the modified cell is a modified human stem cell or a cell obtained from the modified human stem cell, wherein the disruption comprises a deletion of exon 1 of TAP1 gene, and the deletion comprises the nucleic acid sequence ID: 74.

2. The modified cell of claim 1, wherein the modified stem cell has a disruption in an endogenous gene associated with a biosynthesis or transportation pathway of MHC I.

3. The modified cell of claim 2, wherein the disruption comprises a disruption of one or more exons of Transporter associated with antigen presentation 1 (TAP1) gene or a disruption of one or more exons of TAP-associated glycoprotein (TAPBP) gene.

4. The modified cell of claim 3, wherein the disruption of the one or more exons of TAP1 gene comprises a disruption of an exon of TAP1 gene having the nucleic acid sequence ID: 21 or 29.

5. The modified cell of claim 3, wherein the disrupted one or more exons of TAP1 gene comprises one of the nucleic acid sequence IDs: 31-37 and 39-44.

6. (canceled)

7. The modified cell of claim 3, wherein the one or more exons of TAP1 is disrupted such that the one or more exons of TAP1 comprise one of the nucleic acid sequence IDs: 32, 36, 37, 41, 42, 68, 72, 73, 77, and 78.

8. The modified cell of claim 3, wherein the disruption of TAPBP gene comprises a heterozygous disruption of TAP1 gene, and the modified cell expresses wild-type TAPBP gene.

9. The modified cell of claim 3, wherein the disruption of the one or more exons of TAPBP gene comprises a disruption of an exon of TAPBP gene having the nucleic acid sequence ID: 25 or 30.

10. The modified cell of claim 3 wherein the disrupted one or more exons of TAPBP gene comprises one of the nucleic acid sequence IDs: 45-66.

11. The modified cell of claim 3, wherein the one or more exons of TAPBP is disrupted such that the one or more exons of TAPBP comprise one of the nucleic acid sequence IDs: 47, 57, and 58.

12. The modified cell of claim 3, wherein the decreased immunogenicity comprises a decreased level of inflammatory responses induced by the modified cell as compared to the corresponding wild-type cell.

13. The modified cell of claim 3, wherein a karyotype of the modified cell is the same as a karyotype of the corresponding wild-type cell.

14. The modified cell of claim 3, wherein a level of pluripotency of the modified cell is substantially the same as a level of pluripotency of the corresponding wild-type cell.

15. The modified cell of claim 3, wherein the modified human stem cell is selected from the group consisting of totipotent stem cell, a pluripotent stem cell, an embryonic stem cell, an induced pluripotent stem cell, or a multipotent stem cell.

16. The modified cell of claim 3, wherein the modified human stem cell a human embryonic stem cell.

17. A method for improving transplantation of stem cells or cells derived from the stem cells, the method comprising:
culturing in a culture media the modified cells of the claim 3; and
administering a subject a composition comprising the modified cells, wherein immune responses of the subject in response to the transplantation is less than transplantation using wild type stem cells.

18. The method of claim 17, wherein the transplantation is an allogeneic transplantation.

19. The method of claim 17, wherein the immune responses comprise accumulation of T lymphocytes in the subject in response to the transplantation.

20. The method of claim 17, wherein the immune responses comprise accumulation of NK lymphocytes in the subject in response to the transplantation.
